(19) United States
(12) Patent Application Publication
(10) Pub. No.: US 2025/0194481 A1
(43) Pub. Date: Jun. 19, 2025

(54) TOMATO PROPAGATION SYSTEMS AND METHODS FOR BREEDING

(71) Applicant: MJNN LLC, South San Francisco, CA (US)

(72) Inventors: Azize HOMER, South San Francisco, CA (US); Taylor DOHERTY, Pittsburg, TX (US)

(21) Appl. No.: 18/981,231

(22) Filed: Dec. 13, 2024

Related U.S. Application Data
(60) Provisional application No. 63/610,228, filed on Dec. 14, 2023.

(51) Int. Cl. A01H 1/02; A01G 7/04; A01G 22/05; A01G 31/00; A01G 31/02

(52) U.S. Cl. CPC A01H 1/02; A01G 7/045; A01G 22/05; A01G 31/011; A01G 31/025

(57) ABSTRACT

Embodiments of the present disclosure generally relate to systems and methods for plant breeding. More specifically, embodiments of the present disclosure generally relate to apparatus, systems, and methods for tomato plant breeding. In an embodiment, a method includes producing tomatoes from tomato seeds or tomato seedlings in a growing system, wherein the tomato seeds or tomato seedlings are selected to produce at least four generations and harvests of the tomatoes within one calendar year, the growing system comprising a vertical growth column. In another embodiment, a method includes planting tomato seeds or tomato seedlings in growth modules of a vertical tower, the vertical tower being free of fencing and trellising, producing tomato plants and tomatoes from the tomato seeds or tomato seedlings, and harvesting the tomatoes from the tomato plants at a period of 90 days or less from planting the tomato seeds.

### CROSS-REFERENCE TO RELATED APPLICATIONS

This application claims benefit of U.S. provisional patent application Ser. No. 63/610,228, filed Dec. 14, 2023, which is herein incorporated by reference.

### BACKGROUND

### Field

Embodiments of the present disclosure generally relate to systems and methods for plant breeding. More specifically, embodiments of the present disclosure generally relate to apparatus, systems, and methods for tomato plant breeding.

### Description of the Related Art

For hydroponic indoor farming operations, a major contributor to cost of operation is labor, averaging about 50% to about 80% across both small and large farms. Growth habit and canopy size can be important traits to consider when selecting varieties of tomatoes because such characteristics affect labor requirements. Conventional greenhouse fresh market tomato varieties are typically tall, indeterminate types and with high production cost due to labor involved with, for example, trellising and pruning. To date, few, if any, tomato varieties have been bred for indoor growing conditions with LED lighting. Furthermore, tomatoes produced by conventional technologies exhibit undesirably long intervals between seed and harvest. Even with such extended growing times, conventional technologies lack consistent flavor profiles and consistent fruit quality that consumers demand.

There is a need for new and improved apparatus, systems, and methods for tomato plant breeding.

### SUMMARY

In one embodiment, a method of producing tomatoes is provided. The method includes producing tomatoes from tomato seeds or tomato seedlings in a growing system. The tomato seeds or tomato seedlings are selected to produce at least four generations and harvests of the tomatoes within one calendar year and the growing system includes a vertical growth column.

In another embodiment, a method is provided. The method includes planting tomato seeds or tomato seedlings in growth modules of a vertical tower, the vertical tower being free of fencing and trellising, producing tomato plants and tomatoes from the tomato seeds or tomato seedlings, and harvesting the tomatoes from the tomato plants at a period of 90 days or less from planting the tomato seeds.

In another embodiment, a method of producing a tomato seed line is provided. The method includes cross-pollinating a first tomato plant with a second tomato plant to produce a first hybrid offspring tomato seed, growing, in a first tower, the first hybrid offspring tomato seed into a population of first hybrid offspring tomato plants and selecting third plants from the first population based on a first agronomical trait, cross-pollinating the third plants to produce a second hybrid offspring tomato seed, and growing, in a second tower, the second hybrid offspring tomato seed into a population of second hybrid offspring tomato plants and selecting third plants from the second population based on a second agronomical trait. The first towers and second towers are different and the first and second agronomical traits include seeds that produce at least four generations and harvests of tomatoes within one calendar year.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 is a flow chart of a method of an inbred line development cycle, according to embodiments of the disclosure.

FIG. 2A is a schematic perspective view of a controlled environment agriculture system, according to embodiments of the disclosure.

FIG. 2B is a perspective view of a portion of a grow line with grow towers, according to embodiments of the disclosure.

FIG. 3 is a side view of vertical harvesting system, according to embodiments of the disclosure.

FIG. 4 is an end view of a vertical harvesting system, according to embodiments of the disclosure.

FIG. 5A is a graph of the sum of sugar amount for tomatoes produced, according to embodiments of the disclosure.

FIG. 5B is a graph of the sum of organic acids for tomatoes produced, according to embodiments of the disclosure.

FIG. 6 is a graph of exemplary yield data for example dwarf tomatoes produced, according to embodiments of the disclosure.

FIG. 7 is a graph of exemplary Brix data for example dwarf tomatoes produced, according to embodiments of the disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

Embodiments of the present disclosure generally relate to apparatus and methods for tomato plant breeding. The inventors have found new and improved apparatus and methods for tomato plant breeding that can enable harvesting a tomato from a tomato seed in a very short period of time. For example, and in some embodiments, a period from tomato seed to tomato can be about 3 months (90 days) or less, such that four generations per year can be achieved. Conventional technologies are much slower, having a period from tomato seed to tomato of 6 months or more.

Embodiments of the present disclosure also relate to line development of tomato seeds. Conventional seed breeding technologies for line development can take 5-10 years at which point a breeder can field test the breeds and can choose to release one or more lines as varieties or to release a multiline variety. In contrast, embodiments described herein can enable a line development of only 1-2 years. In some examples, embodiments described herein can enable the cross-breeding of four generations of tomato seeds within one year.

The short breeding times can be a consequence of, at least, a unique genetic-mechanical interaction. Here, for example, parameters such as light, temperature, carbon dioxide (CO₂), growth duration, nutrients, the environment, or combinations thereof, among others can be controlled in order to select one or more desirable traits for an offspring. Desirable traits can include architecture (for example, canopy size, growth habit, fruit set, and/or harvestability, among others), yield (for example, flowering habit, fruit size, and/or light use efficiency, among others), and quality (for example, flavor, appearance, texture, and/or disorders, among others).

### Example Line Development and Line Development Cycle

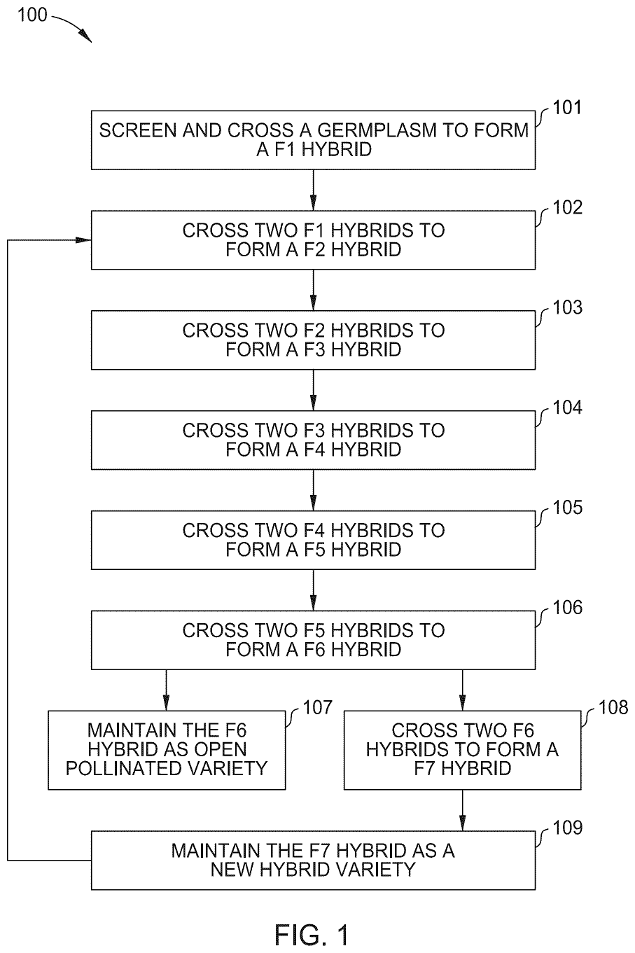

FIG. 1 is a flow chart of a method 100 of an inbred line development cycle. At operation 101, a germplasm is screened and crossed to form a F1 hybrid. Crossing a germplasm involves breeding plants with different genetic material to improve traits and develop new varieties. The cross of the germplasm results in the F1 hybrid seed having a new phenotype than the parental seeds used to form the F1 hybrid. The F1 hybrid is the first generation following a cross. At operation 102, the F1 hybrid is crossed to form an F2 hybrid. To create the F2 hybrid, two members of the F1 hybrid having desired traits are crossed. The F1 hybrid is grown, observed, and members of the F1 hybrid having desired traits are selected for crossing. At operation 103, the F2 hybrid is crossed to form an F3 hybrid. To create the F3 hybrid, two members of the F2 hybrid having desired traits are crossed. The F2 hybrid is grown, observed, and members of the F2 hybrid having desired traits are selected for crossing. At operation 104, the F3 hybrid is crossed to form an F4 hybrid. To create the F3 hybrid, two members of the F3 hybrid having desired traits are crossed. The F3 hybrid is grown, observed, and members of the F3 hybrid having desired traits are selected for crossing. At operation 105, the F4 hybrid is crossed to form an F5 hybrid. To create the F4 hybrid, two members of the F4 hybrid having desired traits are crossed. The F4 hybrid is grown, observed, and members of the F4 hybrid having desired traits are selected for crossing. At operation 106, the F5 hybrid is crossed to form an F6 hybrid. To create the F5 hybrid, two members of the F5 hybrid having desired traits are crossed. The F5 hybrid is grown, observed, and members of the F5 hybrid having desired traits are selected for crossing. At operation 107, the F6 hybrid is maintained as an open pollinated (OP) variety. At operation 108, the F6 hybrid is crossed to form an F7 hybrid. To create the F6 hybrid, two members of the F6 hybrid having desired traits are crossed. The F6 hybrid is grown, observed, and members of the F6 hybrid having desired traits are selected for crossing. At operation 109, the F7 hybrid is maintained as a new hybrid variety. After operation 109, the method 100 may optionally repeat operations 101-109.

Prior to the one or more crossings, desirable characteristics such as architecture, yield, quality (each described above), or combinations thereof, among others, can be selected. Unlike conventional technologies, embodiments described herein utilize, for example, control over various parameters such as light, temperature, carbon dioxide (CO₂), growth duration, nutrients, the environment, or combinations thereof, among others during one or more generations of breeding. Such control can enable the speeding up of the plant growth to reduce the generation time from seed to seed. In some embodiments, the timing of the generations, in relation to the apparatus described herein can enable the improved, and quicker, growth cycle.

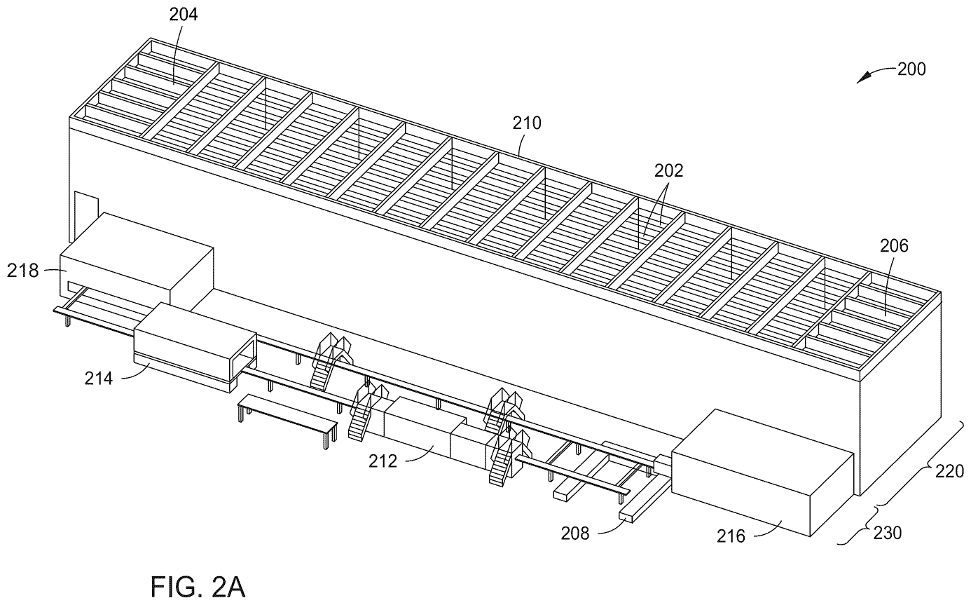

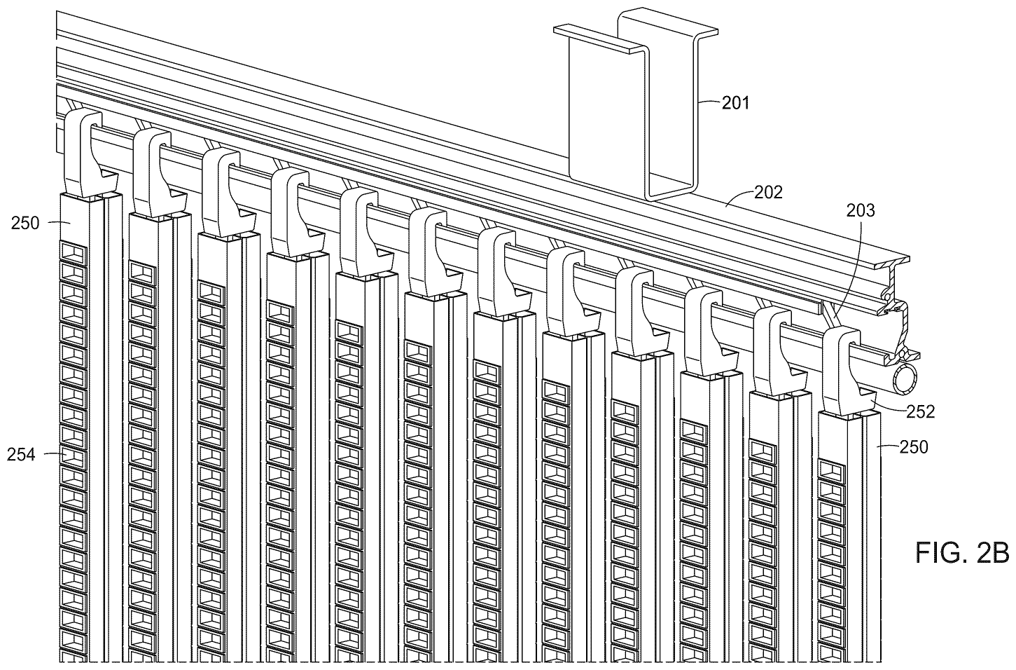

The line development cycle shown in FIG. 1 can be performed in, for example, those systems described below with reference to one or more of FIGS. 2-4. Data and phenotype characteristics of tomato plants and tomatoes produced by embodiments described herein are discussed in the Examples Section. In some cases, line development may refer to the process of obtaining homozygous inbred plants derived from crosses between parental lines. In some cases, line development cycle time may involve recycling breeding material back into a crossing block.

Relative to conventional technologies, tomatoes (and/or tomato plants) produced according to embodiments described herein can have more dense plant canopies with various genotypes (for example, dwarf genotypes); higher plant density per square meter; higher light over the entire canopy; faster (shorter) life cycles with high yields; or combinations thereof. In addition, tomatoes (and/or tomato plants) produced according to embodiments described herein can be grown without trellising, training, pruning, and/or support structure. Accordingly, production costs are substantially reduced relative to conventional technologies.

### Example Apparatus and Systems

Tomatoes and tomato plants can be grown by an apparatus and methods for harvesting vertically grown fruits, plants, or vegetables. In one embodiment, a harvesting system includes a grow line and one or more grow towers coupled to and moveable along the grow line. A plurality of platforms are disposed adjacent to the grow towers and include one or more robots disposed on the platforms. Embodiments of the disclosure also provide for harvesting tools, such as robot end effectors for harvesting one or more types of produce.

FIG. 2A is a schematic perspective view of a controlled environment agriculture system 200. The system 200 can be configured for high-density tomato growth and tomato crop yield. The system 200 includes an environmentally controlled growing chamber 220 and a vertical tower conveyance system 210 disposed within the growing chamber 220. The vertical tower conveyance system 210 is operable to convey grow towers 250 (also referred to as a vertical growth column or vertical tower), described in greater detail with respect to FIG. 2B, with tomato crops/plants therein through the growing chamber 220. The tomato crops or plants grown within the system 200 can exhibit gravitropic, geotropic, and/or phototropic growth characteristics. The system 200 can be configured to grow a single tomato plant type at a time or grow multiple tomato plant types concurrently. The grow towers 250 can be free of fencing, trellising, or other support structure conventionally utilized for tomato and tomato plant growth.

The system 200 also includes additional conveyance systems, such as a central processing system 230, for moving the grow towers 250 in a circuit or pathway within the system 200 throughout the tomato crop or plant growth cycle. The central processing system 230 includes one or more conveyance mechanisms for directing grow towers 250 to stations for loading tomato plant plugs into, and harvesting tomato crops from, the grow towers 250. In this non-limiting example, the central processing system 230 includes a harvester station 208, a washing station 212, and a transplanter station 214. The harvester station 208 removes tomato crops from the grow towers 250 and deposits harvested tomato crops into food-safe containers which may then be conveyed to post-harvest facilities (e.g. preparation, washing, packaging, storage, etc.).

In the illustrated embodiment, various stations of the central processing system 230 operate on grow towers 250 disposed in a horizontal orientation. A pick-up station 218, and associated control logic, includes a robot operable to releasably grasp a grow tower oriented horizontally from a loading location, rotate the grow tower into a vertical orientation, and attach the grow tower to a transfer station for insertion into a selected grow line 202 of the growing chamber 220. At the other end of the growing chamber 220, a laydown station 216, and associated control logic, is operable to releasably grasp and move a vertically oriented grow tower from a buffer region, rotate the grow tower to a horizontal orientation, and position the grow tower on a conveyance system for loading into the harvester station 208. The stations 218, 216 each include a robotic arm, such as a six-degree of freedom robotic arm with end effectors for grasping the grow towers 250.

The growing chamber 220 also includes automated loading and unloading mechanisms for inserting grow towers 250 into selected grow lines 202 and unloading grow towers 250 from the grow lines 202. In one implementation, a load transfer conveyance mechanism 204 includes a powered and free conveyor system that conveys carriages loaded with grow towers 250 from the pick-up station 218 to a selected grow line 202. The load transfer conveyance mechanism 204 also includes one or more actuators that push the grow towers 250 onto a grow line 202. Similarly, an unload transfer conveyance mechanism 206 includes one or more actuators that push or pull the grow towers 250 from the grow lines 202 into a carriage of another powered or free conveyor mechanism, which conveys the carriages from the grow line 202 to the laydown station 216.

The circuit or pathway includes a staging area for loading the grow towers 250 into and out of the vertical tower conveyance system 210. The vertical tower conveyance system 210 within the growing chamber 220 is configured to suspend or otherwise support and translate one or more grow towers 250 along a plurality of grow lines 202. Each grow tower 250 is configured to contain plant growth media that supports a root structure of at least one tomato crop or plant growing therein. The grow towers 250 are releasably attach to the grow lines 202 in a substantially vertical orientation and move along the grow lines 202 during a growth phase of the plant. The vertical tower conveyance system 210 and central processing system 230 are arranged in a production circuit under the control of one or more computing and/or control systems.

The growing chamber 220 includes light emitting sources positioned at various locations along and between the grow lines 202 of the vertical tower conveyance system 210. The light emitting sources can be positioned laterally relative to the grow towers 250 in the grow lines 202 and configured to emit light toward faces of the grow towers 250 that include openings from which the plants grow. In one example, the light emitting sources are light emitting diodes (LED). The light emitting sources are a plurality of LEDs arranged in a bar-like structure which is positioned in a vertical orientation to emit light laterally along an entire length of the grow tower. Multiple LED light bar structures are arranged in the growing chamber 220 along and between the grow lines 202. Other lighting configurations are also contemplated. For example, the LED light bar structures may be arranged horizontally between the grow lines 202. In certain embodiments, the LED light bar structures can be water-cooled.

In some embodiments, the light emitting sources can illuminate the tomato plants, seedlings, etc. That is the light emitting sources are configured to emit electromagnetic radiation. The electromagnetic radiation can include artificial light. The electromagnetic radiation emitted by the light emitting sources can be a wavelength or wavelength range from about 10 nm to about 800 nm, such as about 300 nm to about 770 nm, such as about 400 nm to about 600 nm, though other wavelengths and wavelength ranges are contemplated. The light emitting sources may be light emitting diodes, fluorescent lamps, incandescent lamps, mercury-vapor lamps, lasers, blacklights, or combinations thereof utilizing various lamp types and a filter to facilitate light emission. The light emitting source can be configured to emit the electromagnetic radiation in a controlled direction, such as toward the tomato plant growth modules.

The growing chamber 220 also includes a nutrient supply system configured to supply, for example, an aqueous crop nutrient solution to the tomato crops disposed in the grow towers 250 as the grow towers 250 translate through the growing chamber 220. For example, the nutrient supply system can be configured to direct nutrients to a plurality of tomato plant growth modules. The nutrient supply system provides an aqueous crop nutrient solution to a top of the grow towers 250 and gravity causes the nutrient solution to travel down the vertically-oriented grow towers 250 to the tomato crops disposed along a length of the grow towers 250.

The growing chamber 220 also includes an airflow source which is configured to direct airflow in a direction lateral to growth of the tomato crops and through an under-canopy of each plant to disturb a boundary layer of the under-canopy of the plant. In another implementation, airflow is directed from the top of the canopy or orthogonal to the direction of plant growth.

The growing chamber 220 also includes a control system and associated sensors for regulating at least one growing condition or growing parameter, such as air temperature, airflow velocity, relative air humidity, and a concentration of carbon dioxide gas. The control system may further include sub-systems such as HVAC units, chillers, fans, and associated ducting and air handling apparatus. The control system may further include subsystems for controlling a wavelength and/or an intensity of artificial light. The control system may further include subsystems for controlling a nutrient supply.

The grow towers 250 can include various identifying attributes, such as bar codes or radio frequency identification (RFID) tags, to enable sensing and location detection of each grow tower. The system 200 can include corresponding sensors and programming logic for tracking the grow towers 250 during various stages of the tomato crop production cycle and for controlling one or more conditions of the growth environment. The operation of the controls systems and the length of time the grow towers 250 remain in the growth environment can vary depending on a variety of factors, such as tomato crop type, desired tomato crop maturity, and the like.

In operation, grow towers 250, with newly transplanted tomato crops or seedlings disposed therein, are transferred from the central processing system 230 into the vertical tower conveyance system 210. The vertical tower conveyance system 210 moves the grow towers 250 to predefined positions along respective grow lines 202 within the growing chamber 220 in a controlled manner. Within the growing chamber 220, the tomato crops disposed in the grow towers 250 are exposed to conditions of the growth environments, such as light, temperature, humidity, airflow, CO2 concentration, nutrient supply, etc. These conditions (or parameters) can be controlled or adjusted during tomato and tomato plant growth, and can be static or dynamic during growth.

The control systems of the controlled environment agriculture system 200 are capable of automated or manual adjustments to the growth environment to improve growing conditions and improve various tomato crop attributes, such as tomato crop yields, tomato crop visual appeal, and tomato crop nutrient content (for example, sugars). When the tomato crops are ready for harvesting, the grow towers 250 are transferred from the vertical tower conveyance system 210 to the central processing system 230 for harvesting and other processing operations.

FIG. 2B is a perspective view of a portion of the grow line 202 with the grow towers 250. As illustrated, a plurality of the grow towers 250 are arranged in parallel along the grow line 202. Each grow tower 250 includes a plurality of grow sites 254 (also referred to as tomato plant growth modules) distributed along opposing faces of the grow tower 250. In operation, the transplanter station 214 transplants seedlings into empty grow sites 254 of the grow towers 250 where the seedlings remain and mature until the plant is ready for harvesting. The grow line 202 supports the plurality of grow towers 250 and the grow line 202 is supported by a bracket 201 which is coupled to a superstructure, such as a frame or a facility structure. Hooks 252 couple the grow tower 250 to the grow line 202 and support the grow towers 250 in a vertical orientation as the grow towers 250 are translated along the grow line 202. A conveyance mechanism 203 engages the hooks 252 to enable movement of the grow towers 250 along the grow line 202.

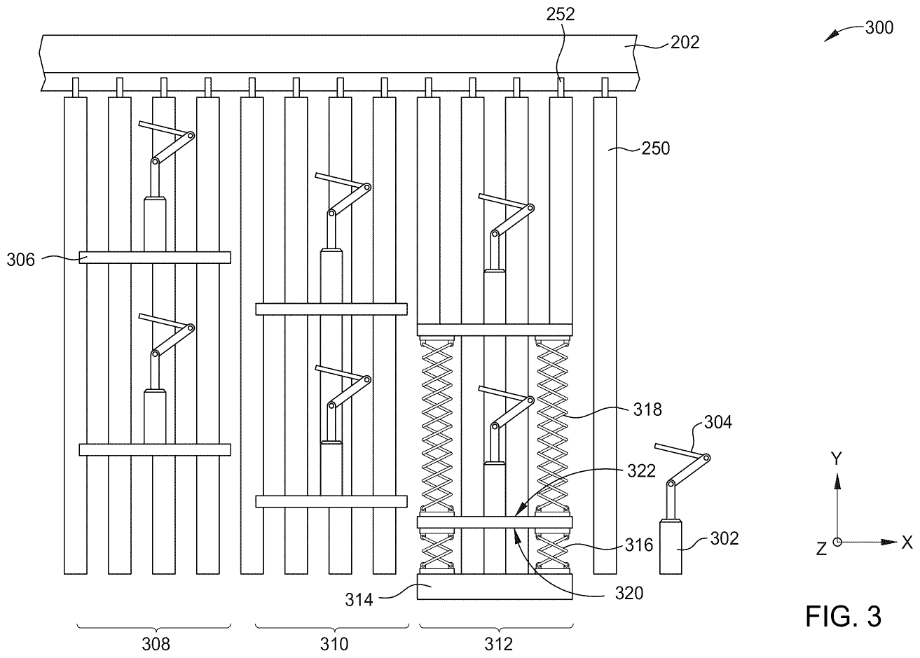

FIG. 3 is a side view of vertical harvesting system 300. The vertical harvesting system 300 includes the grow line 202 and the grow towers 250 coupled to the grow line 202. The vertical harvesting system 300 also includes a plurality of platforms 306 and one or more robots 302 disposed on the platforms 306. In one embodiment, a single robot 302 is disposed on each platform 306. Alternatively, a plurality of robots 302 are disposed on a single platform 306. In operation, grow towers 250 traverse along the grow line 202 in the X-direction and pass across the platforms 306 and the robots 302 which are utilized to harvest plants or fruit from the grow towers 250. While the vertical harvesting system 300 is illustrated as being disposed along a single face or the grow towers 250, the vertical harvesting system 300 may include platforms 306 and robots 302 on the opposing face of the grow towers 250 opposite the illustrated face.

The platforms 306 and/or the robots 302 are moveable relative to the grow towers 250 and/or the grow line 202. In one example, each of the platforms 306 are moveable in the Y-direction. In another example, each of the platforms 306 are moveable in the X-direction. Thus, the platforms 306 can be moved relative to the grow towers 250 to ensure sufficient coverage across the grow towers 250 for harvesting. In one embodiment, the robots 302 disposed on the platforms 306 are fixedly coupled to the platforms 306. In another embodiment, the robots 302 are moveably coupled to the platforms 306. For example, the robots 302 may move in the X-direction along the platforms 306.

The platforms 306 are configured to support the robots 302 thereon and include a first surface 320 and a second surface 322 disposed opposite the first surface 320. In one example, the first surface 320 is considered a bottom surface and the second surface 322 is considered a top surface. The robots 302 are coupled to the second surface 322. In another embodiment, the robots 302 are coupled to the first surface 320. In this embodiment the robots 302 are inverted approximately 180 degree from robots which are disposed on the second surface 322. In another embodiment, the robots 302 are disposed on both the first surface 320 and the second surface 322. In one embodiment, the platforms 306 are a scaffold like structure. While the form factor of the platforms 306 may vary, it is contemplated that any suitable supporting structure may be utilized for the platforms 306. For example, a plurality of cables may be coupled to the platforms 306 and suspend the platforms 306 from an overhead superstructure. In this embodiment, winches or other cable manipulation apparatus are implemented to raise and lower the platforms 306.

In one embodiment, a plurality of harvesting regions 308, 310, 312 are included in the vertical harvesting system 300. For example, a first harvesting region 308 includes one or more platforms 306 and one or more robots 302 disposed on the platforms 306. The platforms 306 and robots 302 of the first harvesting region 308 are positioned adjacent to and harvest plants or fruit from an upper region of the grow towers 250. A second harvesting region 310 includes one or more platforms 306 and one or more robots 302 disposed on the platforms 306. The platforms 306 and robots 302 of the second harvesting region 310 are positioned adjacent to and harvest plants or fruit from a middle region of the grow towers 250. A third harvesting region 312 includes one or more platforms 306 and one or more robots 302 disposed on the platforms 306. The platforms 306 and robots 302 of the third harvesting region 312 are positioned adjacent to and harvest plants or fruit from a lower region of the grow towers 250.

The harvesting regions 308, 310, 312 illustrated are exemplary in nature and a greater or lesser number of harvesting regions may be utilized. While each harvesting region 308, 310, 312 is illustrated with two platforms 306 and robots 302, it is contemplated that a greater or lesser number of platforms 306 and robots 302 may be utilized in each harvesting region 308, 310, 312. In one embodiment, each harvesting region 308, 310, 312 utilizes the same number of platforms 306 and robots 302. Alternatively, one or more of the harvesting regions 308, 310, 312 utilizes a different number of platforms 306 and/or robots 302. In one embodiment, a lowermost region of the grow towers 250 is harvested by a robot 302 which is not disposed on a platform 306, but rather the robot 302 is disposed on a floor of the facility housing the vertical harvesting system 300. Various combinations of platforms 306 and/or robots 302 are contemplated to ensure adequate coverage of the grow towers 250 to facilitate complete and efficient harvesting operations.

In one embodiment, the lowermost platform 306 of each harvest region 308, 310, 312 is coupled to a support base 314 by a first lift 316. The support base 314 is only illustrated in the harvest region 312 for the sake of simplicity. The first lift 316 extends from the support base 314 to the first surface 320 of the lowermost platform 306. A second lift 318 extends from the second surface 322 of the lowermost platform 306 to the first surface 320 of the uppermost platform 306. Additional platforms and lifts are contemplated and may be arranged similar to the illustrated platforms 306 and lifts 316, 318.

While the illustrated embodiment depicts a plurality of lifts 316, 318 coupled to a single platform 306, it is contemplated that each platform 306 may utilize a dedicated lifting mechanism. For example, each platform 306 of each harvesting region 308, 310, 312 are coupled to a respective support base 314 and a lifting mechanism extends between the support base 314 and a single platform 306. In this manner, independent movement of platforms 306 is achieved with less complexity or without impacting the movement of other platforms 306 in the harvesting region 308, 310, 312.

Each lift 316, 318 is operable to raise and lower the platform 306 to which the lift 316, 318 is coupled on the first surface 320. For example, the first lift 316 is operable to raise and lower the lowermost platform 306 while the second lift 318 is operable to raise and lower the uppermost platform 306. The lift 316, 318 are any suitable lifting mechanism capable of raising and lowering the platforms 306 and robots 302 disposed on the platforms 306. Examples of lifting mechanisms include, but are not limited to, scissor-type lifts, telescoping lifts, articulating lifts, screw-type lifts, gear-type lifts, and the like. The lifting mechanism is actuated by hydraulic, electric, mechanical, or pneumatic forces, depending upon the desired implementation. Various combinations of lifting mechanisms may be utilized together to account for gross and fine platform movement or fast and slow platform movement.

Each robot 302 includes an arm 304. The arm 304 extends from the robot 302 and includes an end effector configured to harvest a specific type of plant or fruit from the grow towers 250. In one embodiment, the arm 304 is a six degree of freedom arm, which enables rotation, extension, and other movement in each of the X, Y, and Z-directions. In addition to the elevation or position of the platforms 306, the arm 304 increases the range of harvesting coverage for each robot 302.

In an operational embodiment, each platform 306 and each robot 302 of each harvesting region 308, 310, 312 are positioned in a first harvesting position. The grow towers 250 disposed on the grow line 202 are moved into a harvesting position relative to the harvesting regions 308, 310, 312. Harvesting commences with utilization of the robot 302 and arm 304. Various robot harvesting characteristics, such as the utilization of a boustrophedonic harvesting pattern or the like, ensures complete or substantially complete coverage of the grow towers 250. As harvesting continues and the range of the arm 304 approaches its limits, the platform 306 is either raised or lowered to position the robot 302 within range of unharvested regions of the grow towers 250.

The above sequence is repeated until the predetermined harvesting region (e.g. upper, middle, lower) of the grow tower 250 for each harvesting region 308, 310, 312 is completed. In one embodiment, the grow towers 250 remain stationary during harvesting. Alternatively, the grow towers 250 are moved in the X-direction during harvesting. In this embodiment, the grow towers 250 are moved continuously or in a step-wise fashion. The speed of grow tower movement is calibrated to the harvesting efficiency of each robot to ensure substantially all areas of the grow towers 250 are harvested.

In one embodiment, certain regions of the grow towers 250 are harvested exclusively by a robot 302 in a single harvesting region 308, 310, 312. In another embodiment, certain regions are harvested by a plurality of robots 302 in a plurality of harvesting regions 308, 310, 312. For example, middle regions of the grow towers 250 may be harvested by each of the first, second, and third harvesting regions 308, 310, 312, respectively.

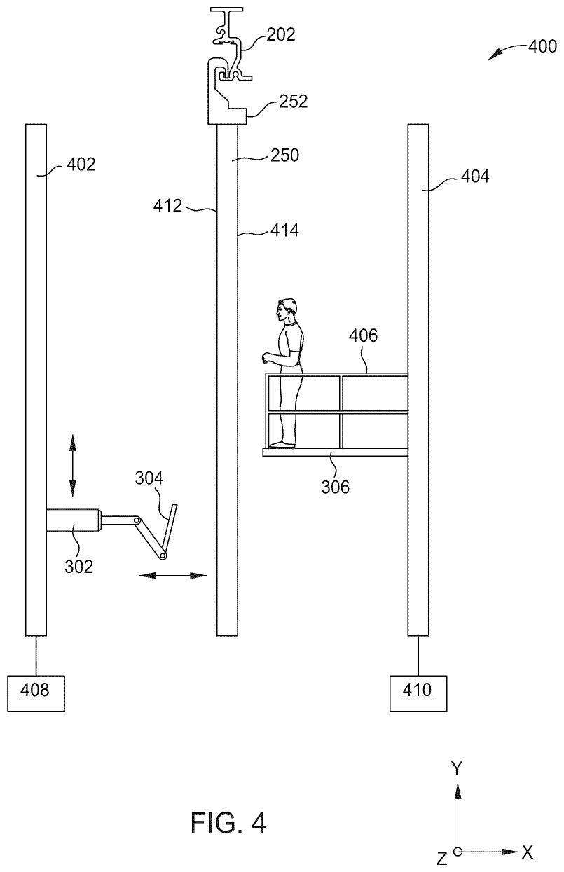

FIG. 4 is an end view of a vertical harvesting system 400. The vertical harvesting system 400 includes a first support column 402 and a second support column 404. The first support column 402 is disposed adjacent to a first face 412 of the grow tower 250 and the second support column 404 is disposed adjacent to a second face 414 of the grow tower 250. The support columns 402, 404 extend vertically with a magnitude similar to the magnitude of the grow tower 250.

In one embodiment, a robot 302 is coupled to the first support column 402. The robot 302 is coupled to the first support column 402 such that the arm 304 is oriented away from the first support column 402 and toward the first face 412 of the grow tower 250. The first support column 402 includes a lifting mechanism similar to those discussed with regard to FIG. 3. In one embodiment, the lifting mechanism is internal to the first support column 402. In another embodiment, the lifting mechanism is external to the first support column 402. A first actuator 408 is coupled to the lifting mechanism of the first support column 402 and the first actuator 408 is configured to move the robot 302 vertically along the first support column 402 in the Y-direction. The first actuator 408 is any suitable type of actuator, such as a mechanical, electric, hydraulic, or pneumatic actuator or the like. In this manner, the robot 302 is raised and lowered along the first face 412 of the grow tower 250 to enable harvesting of plants or fruit from the grow tower 250.

While only a single first support column 402 is illustrated, it is contemplated that a plurality of first support columns 402 are disposed along the grow line 202. Similarly, while only a single robot 302 is illustrated, it is contemplated that a plurality of robots 302 may be coupled to the first support column 402. In another embodiment, one or more robots 302 are coupled to the second support column 404. In this embodiment, the arm 304 of the robots 302 is oriented away from the second support column 404 and toward the second face 414 of the grow tower 250.

In the illustrated embodiment, the platform 306 is coupled to the second support column 404. The platform 306 extends from the second support column 404 toward the second face 414 of the grow tower 250. The platform 306 is sized to support a person performing harvesting tasks or one or more robots 302 disposed on the platform 306. In embodiments utilizing people to perform harvesting tasks, safety railing 406 or other safety and harvesting equipment are coupled to the platform 306.

Similar to the first support column 402, the second support column 404 includes a lifting mechanism which is either internal or external to the second support column 404. A second actuator 410 is coupled to the lifting mechanism of the second support column 404 and the second actuator 410 is configured to move the platform 306, and thus the person or robot 302 on the platform 306, in the Y-direction. The second actuator 410 is any suitable type of actuator, such as a mechanical, electric, hydraulic, or pneumatic actuator or the like. In this manner, the platform is raised and lowered along the second face 414 of the grow tower 250 to enable harvesting of plants or fruit from the grow tower 250.

While only a single second support column 404 is illustrated, it is contemplated that a plurality of second support columns 402 are disposed along the grow line 202. Similarly, while only a single platform 306 is illustrated, it is contemplated that a plurality of platforms 306 may be coupled to the second support column 404. The robots 302 or platforms 306 (which may include robots 302 disposed thereon) may be utilized in any desired configuration with the support columns 402, 404.

In one operational embodiment, the grow towers 250 traverse the grow line 202 while the support columns 402, 404 remain stationary. In this embodiment, the grow towers 250 move continuously or in a step-wise manner. In another operational embodiment, the support columns 402, 404 move relative to the grow towers 250, which are either moving or stationary.

While the embodiments illustrated with respect to FIGS. 3 and 4 are described as being implemented in a plant growing system with vertically oriented towers, it is contemplated that the horizontally oriented grow apparatus disposed in a vertically stacked architecture may also benefit from the embodiments described herein.

### Example Methods

One or more of the systems and apparatus described above with respect to FIGS. 2-4 can be utilized to produce tomatoes. Such systems can be used to produce desired tomato seeds (via, for example, embodiments of FIG. 1). Such systems and apparatus can also be used to produce tomatoes from tomato seedlings and/or tomato seeds. For example, tomato seedlings and/or tomato seeds can be disposed within a grow site 254 (a tomato plant growth module). Various parameters can be controlled or adjusted, statically and/or dynamically, throughout a growth cycle from the seeds and/or seedlings to a tomato plant fruiting tomatoes.

As described above, a plurality of grow sites 254 (tomato plant growth modules) are positioned within a grow tower 250 (a vertical growth column) of a growing system (for example, agriculture system 200). The grow tower is positioned within an environmentally-controlled growing chamber (for example, growing chamber 220). The growing system can include, for example, one or more light emitting sources (for example, an LED) configured to emit artificial light toward the grow sites 254 and a nutrient supply system configured to direct nutrients to the plurality of grow sites 254. In at least one embodiment, the one or more light emitting sources is configured to emit electromagnetic radiation having a wavelength or wavelength range in the ultraviolet region. For example, the light emitting source can be configured to emit electromagnetic radiation having a wavelength or wavelength range from about 10 nm to about 400 nm, though other wavelengths and wavelength ranges are contemplated.

During a tomato plant production method one or more growing conditions can be changed or adjusted. Such growing conditions can include a wavelength (or wavelength of artificial light), an intensity of artificial light, a temperature, a concentration of carbon dioxide, a nutrient supply, and airflow value, a relative humidity value, a light duration extension value (e.g., the duration of time during which a plant is exposed to light, photoperiod extension), or combinations thereof.

The tomato seeds and/or tomato seedlings utilized for embodiments described herein can be produced via the line development cycle shown in FIG. 1. The tomato seeds and/or tomato seedlings used for embodiments described herein have one or more of the following characteristics:
(a) The tomato seeds and/or tomato seedlings can produce three or more generations and harvests of tomatoes within one calendar year, such as four or more generations and harvests of tomatoes within one calendar year, such as five or more generations and harvests within one calendar year. Any of the foregoing numbers can be used singly to describe an open-ended range or in combination to describe a close-ended range.
(b) The tomato seeds and/or tomato seedlings can produce tomatoes that can be harvested from tomato plants at a period of about 120 days or less, such as about 100 days or less, such as about 90 days or less, and/or about 70 days or more, about 80 days or more, about 90 days or more, or combinations thereof, though other values are contemplated. Any of the foregoing numbers can be used singly to describe an open-ended range or in combination to describe a close-ended range.
(c) The tomato seeds and/or tomato seedlings produce tomato plants, and the number of tomato plants that can be produced per square meter of the growing system is about 5 or more, about 10 or more, about 15 or more, about 20 or more, or about 30 or more, and/or about 50 or less, about 40 or less, or about 30 or less, or combinations thereof, though other values are contemplated. Any of the foregoing numbers can be used singly to describe an open-ended range or in combination to describe a close-ended range.
(d) The tomato seeds and/or tomato seedlings produce tomatoes having a Brix value (or a mean Brix value) of about 1 or more, about 2 or more, about 3 or more, about 4 or more, about 5 or more, about 6 or more, about 7 or more, about 8 or more, about 9 or more, about 10 or more, about 11 or more, about 12 or more, though other values are contemplated. Any of the foregoing numbers can be used singly to describe an open-ended range or in combination to describe a close-ended range.
(e) The tomato seeds and/or tomato seedlings can produce tomatoes having a cumulative total yield of tomatoes harvested per tomato plant that can be about 400 g/plant or more, about 500 g/plant or more, about 600 g/plant or more, about 700 g/plant or more, and/or about 1,100 g/plant or less, about 1,000 g/plant or less, about 900 g/plant or less, about 800 g/plant or less, or combinations thereof, though other values are contemplated. Any of the foregoing numbers can be used singly to describe an open-ended range or in combination to describe a close-ended range.
(f) The tomato seeds and/or tomato seedlings can produce a total number of tomatoes harvested per tomato plant that can be about 90 tomatoes/plant or more, about 100 tomatoes/plant or more, about 110 tomatoes/plant or more, about 120 tomatoes/plant or more, about 130 tomatoes/plant or more, about 140 tomatoes/plant or more, and/or about 190 tomatoes/plant or less, about 180 tomatoes/plant or less, about 170 tomatoes/plant or less, about 160 tomatoes/plant or less, about 150 tomatoes/plant or less, about 140 tomatoes/plant or less, about 130 tomatoes/plant or less, about 120 tomatoes/plant or less, or combinations thereof. Any of the foregoing numbers can be used singly to describe an open-ended range or in combination to describe a close-ended range.
(g) The tomato seeds and/or tomato seedlings can produce a tomato plant that can be characterized as yielding, on average, about 300 g of tomatoes/m2/day or more, about 380 g of tomatoes/m2/day or more, about 400 g of tomatoes/m2/day or more, about 500 g of tomatoes/m2/day or more, about 600 g of tomatoes/m2/day or more, about 700 g of tomatoes/m2/day or more, and/or about 900 g of tomatoes/m2/day or less, about 800 g of tomatoes/m2/day or less, about 700 g of tomatoes/m2/day or less, about 600 g of tomatoes/m2/day or less, or combinations thereof. Any of the foregoing numbers can be used singly to describe an open-ended range or in combination to describe a close-ended range.

The aforementioned characteristics can change depending on the tomato type such as small cherry tomato, medium cherry tomato, large cherry cocktail tomato, small roma tomato, large roma/plum tomato, small slider (small beef) tomato, among others. Other tomato types are contemplated.

The following examples are put forth so as to provide those of ordinary skill in the art with a complete disclosure and description of how to make and use embodiments of the present disclosure, and are not intended to limit the scope of embodiments of the present disclosure. Efforts have been made to ensure accuracy with respect to numbers used but some experimental errors and deviations should be accounted for.

### EXAMPLES

In one application of method 100, the pedigree selection begins when about 250 to about 500 seedling populations (e.g., an F2 hybrid strain plant) are grown, selected and transplanted as dwarf seedlings. The pedigree selection continues with the evaluation of about 20 to about 40 dwarf plants grown from the dwarf seedlings. A subset of the dwarf plants are selected for further breeding based on criteria described herein. The progeny of the selected dwarf plants (e.g., progeny producing an F3 hybrid strain plant) are further selected based on promising characteristics. From each progeny selection, about 10 to about 20 plants are grown per progeny plant family. Finally, about 10 to about 20 F4 hybrid strain plants are selected from the cultivated F3 hybrid strain plants. The F4 hybrid strain plants are advanced to an F6 hybrid strain. During advancement, plant families that do not meet breeding objectives are eliminated. In some cases, replicated trials may be implemented during F5 hybrid strain and F6 hybrid strain generations, depending on at least the degree of uniformity present in the generations. In some cases, the pedigree selection process may take about 2 years.

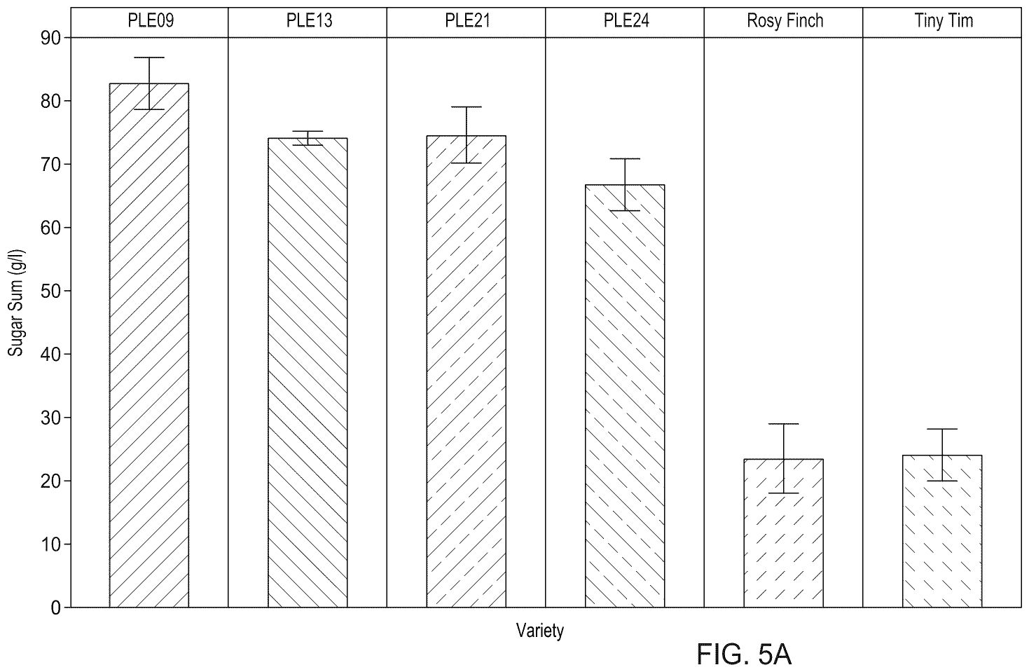

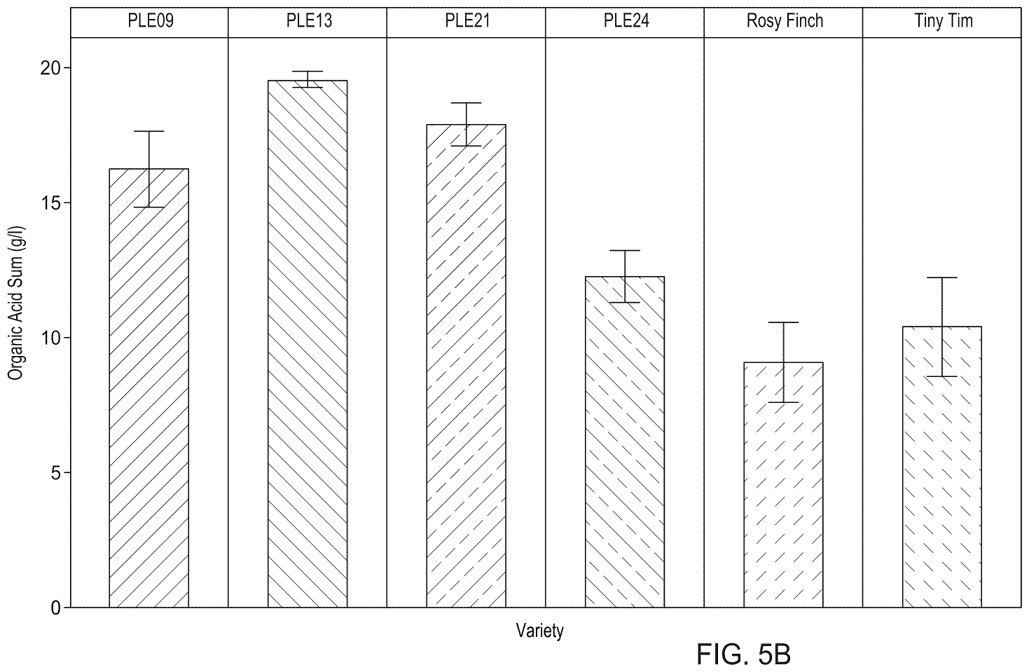

FIG. 5A is a graph of the sum of sugar amount for tomatoes produced. The sum of sugar amount (in grams per liter, g/L) for tomatoes produced by embodiments described herein (red, green, blue, and orange bars) is compared against two conventional comparative examples (teal and purple bars). Sugars were analyzed via high pressure ion chromatography (IC), mass spectrometry (MS) using a Dionex™ CarboPac™ SA10-Fast-4 μm 2 mm×250 mm IC Column. The sum of sugar amount takes into account the following carbohydrates: sucrose, galactose, glucose, xylose, fructose, and maltose. The results indicate that embodiments described herein have, at least, a unique flavor profile and higher levels of sugars/carbohydrates than the comparative examples.

FIG. 5B is a graph of the sum of organic acids for tomatoes produced. The sum of organic acids (g/L) for tomatoes produced by embodiments described herein (red, green, blue, and orange bars) is compared against two comparative conventional examples (teal and purple bars). Organic acids were analyzed via high pressure IC-MS using a Dionex™ IonPac™ AS11-HC-4 μm 2 mm×250 mm IC Column. The sum of organic acids takes into account the following organic acids: quinic acid, acetic acid, malic acid, ascorbic acid, oxalic acid, citric acid, and isocitric acid. The results indicate that embodiments described herein have, at least, a larger sum of organic acids than the comparative examples.

Dwarf phenotypes were grown in a fast, short life-cycle (about 80-90 days). The tomatoes can be grown in a compact manner, with dense plant canopies, and high plant density per square meter. Different fruit colors of tomatoes can also be selected, among other phenotypes. The tomatoes and tomato plants can be grown without trellising, training, pruning, and/or support structure. Accordingly, production costs are substantially reduced relative to conventional technologies.

Yellow tomatoes were determined to have a mean Brix value of about 10 and the red tomatoes were determined to have a mean Brix value of about 11. The embodiments described herein enable the production of dwarf, compact tomato lines with different fruit colors and sweeter flavor than commercially available microdwarf tomato varieties. As further described below, the Brix values determined are substantially higher than commercial varieties which have a Brix value of less than 6.

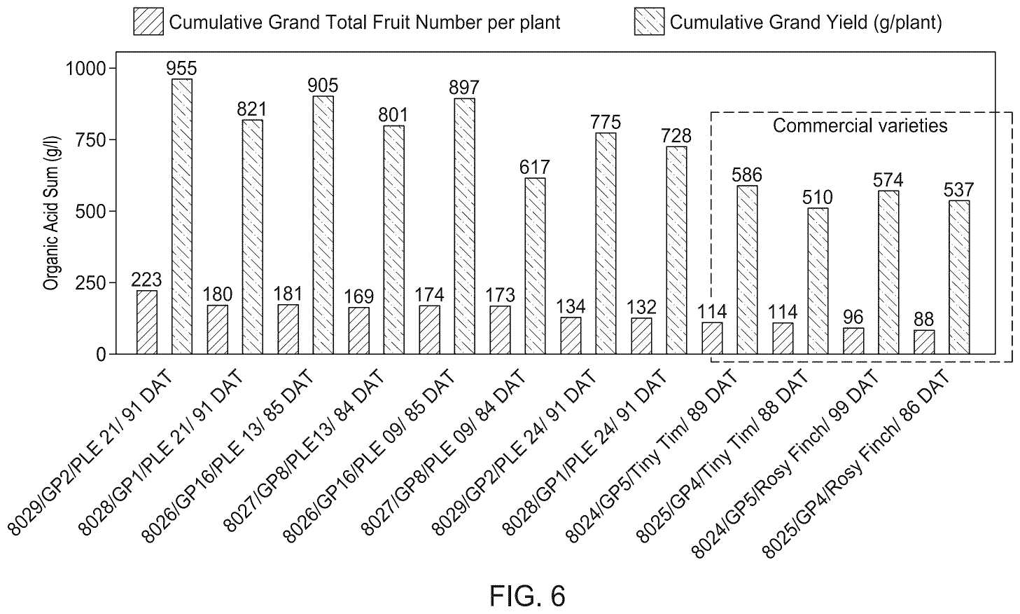

FIG. 6 is a graph of exemplary yield data for example dwarf tomatoes produced. The example dwarf tomatoes produced according to embodiments described herein are compared against 4 comparative conventional example tomatoes (commercial varieties). The data presented shows a yield comparison for cumulative total yield (g/plant) shown by the red bars and cumulative total fruit per plant (number/plant) shown by the blue bars. While commercial dwarf varieties produced a cumulative total yield of between 500 g/plant and 586 g/plant, dwarf varieties produced by embodiments described herein had significantly higher cumulative total yields, ranging from about 617 g/plant to about 965 g/plant. In addition, commercial dwarf varieties produced a cumulative total fruit per plant of between 88 fruit/plant and 114 g/plant. In contrast, dwarf varieties produced by embodiments described herein were determined to have a significantly higher cumulative total fruit per plant, ranging from about 132 g/plant to about 223 g/plant.

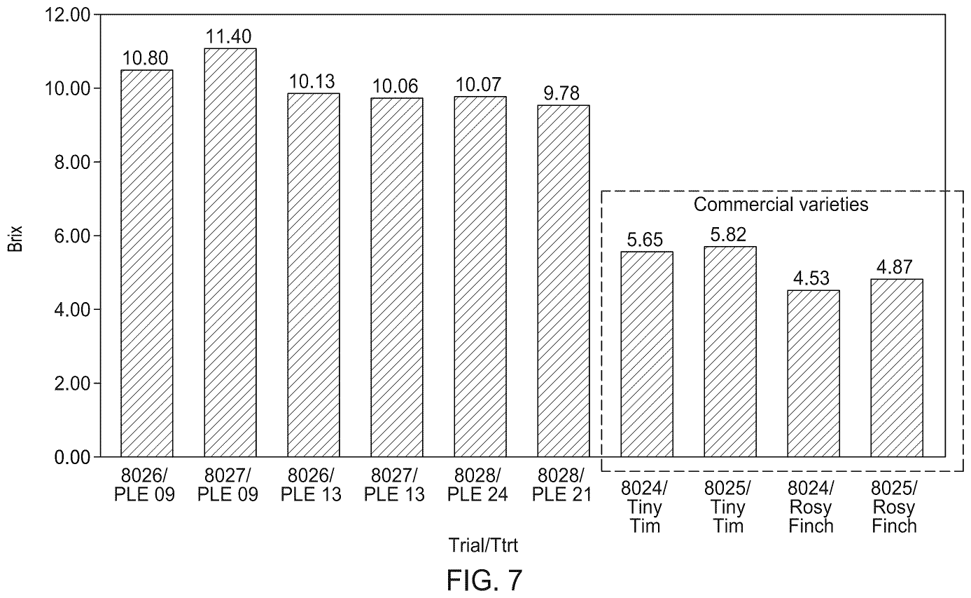

FIG. 7 is a graph of exemplary Brix data for example dwarf tomatoes produced. The example dwarf tomatoes produced according to embodiments described herein are compared against 4 comparative conventional example tomatoes (commercial varieties). The data presented shows Brix values, with higher Brix values indicating a sweeter taste. Embodiments described herein enabled production of tomatoes having a mean Brix value ranging between about 9.7 and about 11.4 (first six bars of the chart). In contrast, the mean Brix value of commercial varieties (last four bars of the chart) were significantly lower—no higher than 5.8.

A conventional high-wire method of growing tomatoes with tall-indeterminate characteristics incurs high labor costs associated with trellising, fencing, pruning, and/or support structures are required. In contrast, an example tomato plant produced by embodiments described herein has characteristics such as dwarf-determinate and compact canopy. In further contrast to conventional technologies, embodiments described herein enable production of tomatoes without trellising, fencing, pruning, and/or support structures.

As is apparent from the foregoing general description and the specific aspects, while forms of the aspects have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, process operation, process operations, an element or a group of elements is preceded with the transitional phrase “comprising,” it is understood that we also contemplate the same composition or group of elements with transitional phrases “consisting essentially of,” “consisting of,” “selected from the group of consisting of,” or “Is” preceding the recitation of the composition, process operation, process operations, element, or elements and vice versa, such as the terms “comprising,” “consisting essentially of,” “consisting of” also include the product of the combinations of elements listed after the term.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

References cited herein are incorporated by reference herein in their entirety to indicate the state of the art as of their publication or filing date and it is intended that this information can be employed herein, if needed, to exclude specific embodiments that are in the prior art.

As used herein, the indefinite article “a” or “an” shall mean “at least one” unless specified to the contrary or the context clearly indicates otherwise. For example, embodiments comprising “a tomato” include embodiments comprising one, two, or more tomatoes, unless specified to the contrary or the context clearly indicates only one tomato is included.

While the foregoing is directed to embodiments of the present disclosure, other and further embodiments of the disclosure may be devised without departing from the basic scope thereof, and the scope thereof is determined by the claims that follow.

What is claimed is:

1. A method of producing tomatoes, comprising:
producing tomatoes from tomato seeds or tomato seedlings in a growing system, wherein the tomato seeds or tomato seedlings are selected to produce at least four generations and harvests of the tomatoes within one calendar year, the growing system comprising a vertical growth column.

2. The method of claim 1, wherein the vertical growth column is positioned within an environmentally-controlled growing chamber, the vertical growth column comprising a plurality of vertically aligned tomato plant growth modules.

3. The method of claim 2, wherein the growing system further comprises:
a light emitting source configured to emit artificial light toward the tomato plant growth modules; and
a nutrient supply system configured to direct nutrients to the plurality of vertically aligned tomato plant growth modules.

4. The method of claim 3, wherein the light emitting source comprises a light emitting diode.

5. The method of claim 4, wherein the light emitting source is configured to emit electromagnetic radiation having a wavelength or wavelength range from about 10 nm to about 400 nm.

6. The method of claim 5, wherein:
the tomato seeds or tomato seedlings produce tomato plants; and
a number of tomato plants produced per square meter of the growing system is about 20 or more.

7. The method of claim 6, wherein the tomatoes produced have a brix value of about 6 or more.

8. The method of claim 7, wherein the tomatoes produced have a brix value of about 8 or more.

9. The method of claim 8, further comprising:
adjusting one or more growing conditions during the producing the tomatoes, the one or more growing conditions comprising:
a wavelength of artificial light;
a duration of artificial light exposure;
an intensity of artificial light;
a temperature;
a concentration of carbon dioxide;
a nutrient supply;
a relative humidity;
an airflow;
or combinations thereof.

10. A method, comprising:
planting tomato seeds or tomato seedlings in growth modules of a vertical tower, the vertical tower being free of fencing and trellising;
producing tomato plants and tomatoes from the tomato seeds or tomato seedlings; and
harvesting the tomatoes from the tomato plants at a period of 90 days or less from planting the tomato seeds.

11. The method of claim 10, further comprising:
changing one or more growing conditions during the producing of the tomato plants and tomatoes from the tomato seeds or tomato seedlings.

12. The method of claim 11, wherein the one or more growing conditions comprises:
a wavelength of artificial light;
an intensity of artificial light;
a temperature;
a concentration of carbon dioxide;
a nutrient supply; or combinations thereof.

13. The method of claim 12, wherein the tomatoes harvested are characterized as having a brix value of about 6 or more.

14. The method of claim 13, wherein the tomatoes harvested are characterized as having a brix value of about 8 or more.

15. The method of claim 14, wherein a cumulative total yield of tomatoes harvested per tomato plant is about 500 g/plant or more.

16. The method of claim 15, wherein a total number of tomatoes harvested per tomato plant is about 90 tomatoes/plant or more.

17. The method of claim 16, wherein each tomato plant is characterized as yielding, on average, about 380 g to about 500 g of tomatoes/m²/day or more.

18. A method of producing a tomato seed line, the method comprising:
cross-pollinating a first tomato plant with a second tomato plant to produce a first hybrid offspring tomato seed;
growing, in a first tower, the first hybrid offspring tomato seed into a first population of first hybrid offspring tomato plants and selecting third plants from the first population based on a first agronomical trait; and
cross-pollinating the third plants to produce a second hybrid offspring tomato seed;
growing, in a second tower, the second hybrid offspring tomato seed into a second population of second hybrid offspring tomato plants and selecting third plants from the second population based on a second agronomical trait, wherein:
the first towers and second towers are different; and
one or the first and second agronomical traits comprises seeds that produce at least four generations and harvests of tomatoes within one calendar year.

19. The method of claim 18, comprising:
one of the first agronomical traits or second agronomical traits comprises seeds that produce at least one tomato within 90 days.

20. The method of claim 19, wherein the agronomical traits further comprise seeds that produce tomatoes having a Brix value of about 6 or more.
